(19) United States
(12) Patent Application Publication
(10) Pub. No.: US 2014/0349313 A1
(43) Pub. Date: Nov. 27, 2014

(54) DATA ANALYSIS METHODS UTILIZING PHENOTYPIC PROPERTIES

(71) Applicant: Becton, Dickinson and Company, Franklin Lakes, NJ (US)

(72) Inventors: Robert Balderas, San Diego, CA (US); Mirko Corselli, San Diego, CA (US)

(21) Appl. No.: 14/265,240

(22) Filed: Apr. 29, 2014

Related U.S. Application Data
(60) Provisional application No. 61/817,430, filed on Apr. 30, 2013.
(60) Provisional application No. 61/931,457, filed on Jan. 24, 2014.

(51) Int. Cl. G01N 33/569; G06F 19/14

(52) U.S. Cl. CPC G01N 33/56966; G06F 19/14
USPC 435/7.24; 435/287.2; 702/21

(57) ABSTRACT

The present invention provides a method of identifying sub-populations of cells in a cellular sample. Aspects of the method include categorizing cells of the cellular sample into at least a first and second population based on a first phenotypic property. The method may further include sub-categorizing each of the first and second population into sub-populations of cells based on a second and third phenotypic property, e.g., by using X detectable labels providing Y distinct signals, wherein X>Y, to identify sub-populations of cells in the cellular sample.

### CROSS-REFERENCE TO RELATED APPLICATIONS

Pursuant to 35 U.S.C. §119 (e), this application claims priority to the filing dates of the U.S. Provisional Patent Application Ser. No. 61/817,430, filed Apr. 30, 2013 and U.S. Provisional Patent Application Ser. No. 61/931,457 filed on Jan. 24, 2014, the disclosures of which are herein incorporated herein by reference.

### INTRODUCTION

Today, one of the most powerful tools for immunophenotypic study of the immune system is polychromatic flow cytometry. Over the last decade, knowledge of the immune system has greatly increased, partly due to the development of flow cytometry. Cell populations that were considered to be homogenous in the past now appear complex. The identification of specialized lymphocyte subsets such as naïve, memory, or cytotoxic T lymphocytes or monocyte subsets has considerably helped the general understanding of immunopathogenesis during HIV and SIV infection. Moreover, the polychromatic flow cytometry technique has become increasingly useful in identifying rare subsets of cells such as DC, where a minimum of eight fluorescent parameters, in addition to the physical parameters such as forward scatter (FSC) and side scatter (SSC), are ideal to distinguish five nonoverlapping DC subsets simultaneously.

Despite access to commercially available flow cytometers that can measure up to 12 colors without significant modifications, a limited number of laboratories are routinely using such instruments. Although developing a reliable multicolor panel is time consuming and requires a number of validation trials, compared to 2 to 4-color assays, the amount of information provided by such a panel will aid in the development of further understanding of the immune system, potentially defining cell subsets that might otherwise be missed. In addition, using a multicolor flow cytometry panel can decrease the amount of blood needed for immunophenotyping, which is often limited especially during longitudinal studies. Until recently, most research laboratories were measuring populations of lymphocytes, monocytes, and DC using separate antibody panels in individual tubes, mainly because of technical limitations. With advances of the flow cytometry technology, scientists are now able to measure up to 17 colors in one single panel.

### SUMMARY

The present invention provides a method of identifying sub-populations of cells in a cellular sample. Aspects of the method include categorizing cells of the cellular sample into at least a first and second population based on a first phenotypic property. The method may further include sub-categorizing each of the first and second population into sub-populations of cells based on a second and third phenotypic property, e.g., by using X detectable labels providing Y distinct signals, wherein X>Y, to identify sub-populations of cells in the cellular sample.

In some instances, the first phenotypic property may be a physical property of the cell. The first phenotypic property may be cell autofluorescence, cell granularity as identified using side scatter (SSC), or cell size as identified using forward scatter (FSC), side scatter (SSC), axial light loss (ALL) or a combination thereof. In some instances, the first phenotypic property may be expression of a cellular marker. Expression of a cellular marker may be identified using a detectable label that specifically binds to the cellular marker

In some instances, a method for distinguishing at least four cell surface marker populations in a sample using three detectable signals is provided, where embodiments of the methods include providing at least two detectable labels in which a first label has a specificity for a first cellular marker on a first cell type and a second label has a specificity for a second cellular marker on a second cell type, providing a third detectable label that has a specificity for a third cellular marker, providing a fourth detectable label that has a specificity for a fourth cellular marker, wherein the third and fourth detectable labels provide substantially the same signal and wherein the third cellular marker is found on a sub-population of the first cell type and the fourth cellular marker is found on a sub-population of the second cell type. The labels may be combined with a single sample, three signals may be detected and at least four cellular makers may be distinguished from three signals detected.

Distinguishing the four cellular markers may include establishing a first cell population gate that will contain the first cell type within the gate and detecting the third label within the first gate. Next, a second cell population gate may be established that will contain the second cell type within the gate and the fourth label may be detected within the second gate. The third and fourth label may produce the same signal and may be distinguished by the gating of the first and second cell population gate.

A method of distinguishing a number X of primary and secondary cellular markers in a sample from a number Y of primary and secondary detectable signals is disclosed that includes providing at least two detectable primary labels specific for cellular markers wherein each detectable primary label is specific for one cell type and each provides a distinct detectable signal. Next, providing at least two detectable secondary labels specific for cellular markers present in sub-populations of the cell types, wherein the secondary labels provide a number of distinct detectable signals, and wherein the total number detectable secondary labels exceeds the number of distinct detectable signals from the secondary labels. After combining the primary and secondary labels with a single sample a number Y of detectable signals may be detected from a number X of cellular labels wherein X>Y.

A number X of cellular markers in the sample may be distinguished from the Y number of signals wherein X>Y by detecting a first distinguishable signal from a first label corresponding to a first cell type and establishing a first cell population gate that contains a first cell type within the gate. Next detect a second distinguishable signal from a second label corresponding to a second cell type and establish a second cell population gate that contains that cell type. Detect a third signal from a third label specific for a first sub-population within the first gate and distinguish it from a fourth signal from a fourth label specific for a second sub-population within the second gate wherein the third and fourth label provide an identical signal and wherein the third and fourth label are distinguished by the gating of the first and second cell population gate.

In some embodiments the detectable labels are fluorescently labeled antibodies. In some embodiments the primary cell types may be NK cells, T cells, monocytes, B-cells, Macrophage, Dendritic cell, Neutrophil, Eosinophil and Basophils or any combination thereof. In some embodiments the cell-type subpopulations may be identified by any cell surface markers such as CD4, CD8, CD45, CD25, CD 27, and or CCR7. In some embodiments a data processing unit implements the step of distinguishing the cellular markers. Detecting the signals may comprise flow cytometrically analyzing the sample.

A method for distinguishing a number of primary and secondary cellular markers X in a sample from a number of primary and secondary detectable signals Y, is disclosed that includes processing a single sample with a flow cytometer to obtain a multidimensional data set that contains at least two distinguishable signals from at least two primary labels for cellular markers for at least two cell types and at least one distinguishable signal from at least two secondary labels specific for at least two cellular markers present in sub-populations of the cell types. The total number of secondary labels exceeds the number of distinguishable signals from the secondary labels. The data set may be stored in a machine readable memory; and the data set may be operated on to distinguish a number of cellular markers X in the sample using a number of signals Y wherein X>Y.

In some embodiments the operation may include establishing a first cell population gate that contains a first cell type within the gate, detecting a first label specific for a sub-population within the gate, establishing a second cell population gate that contains a second cell type within the gate and detecting a second label specific for a sub-population within the gate wherein the first and second label provide an identical signal and wherein the first and second label are distinguished by the gating of the first and second cell population gate.

A system of this invention may include a flow cytometer configured to produce a data set, a data processing unit and a memory storing a marker deconvolution system comprising a data program code for execution by the processing unit wherein the program code is configured to transform the data set from a number of signal sets, X to a number of marker density data sets, Y wherein Y>X.

### BRIEF DESCRIPTION OF THE FIGURES

FIG. 1 depicts flow chart describing steps of an embodiment of this invention.

FIG. 2 shows density plots illustrating gating steps of this invention.

FIG. 3 shows density plots illustrating gating steps of this invention.

FIG. 4 shows density plots illustrating gating steps of this invention.

FIG. 5 shows density plots illustrating gating steps of this invention.

FIG. 6 shows density plots illustrating gating steps of this invention.

FIG. 7 shows density plots illustrating gating steps of this invention.

FIG. 8 shows density plots illustrating the use of light scatter properties in distinguishing cell types.

FIG. 9 shows density plots illustrating the use of cellular autofluorescence in distinguishing cell types.

FIG. 10 shows density plots illustrating the use of light scatter properties and cell signature in distinguishing cell types.

FIG. 11 shows density plots illustrating further characterization of cell types distinguished by light scatter and cell signature.

FIG. 12 shows density plots illustrating phenotypic and functional differences cell types derived from different tissues.

### DETAILED DESCRIPTION

Methods of identifying sub-populations of cells in a cellular sample are provided. Aspects of the methods include categorizing cells of the cellular sample into at least a first and second population based on a first phenotypic property. The method may further include sub-categorizing each of the first and second populations into sub-populations of cells based on a second and third phenotypic property using X detectable labels providing Y distinct signals, wherein X>Y, to identify sub-populations of cells in the cellular sample. Also provided are systems and kits that find us in practicing the subject methods.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing embodiments of the invention, aspects of embodiments of the methods will be described first in greater detail. Next, embodiments of systems and kits that may be used in practicing methods of the invention are reviewed.

### Methods

As summarized above, embodiments of the invention are directed to methods of identifying sub-populations of cells in a cellular sample. Aspects of the methods include categorizing cells of the cellular sample into primary populations based on one or more phenotypic properties. The term “phenotypic property” is used broadly to refer to any observable physical or biochemical characteristics of a cell or markers expressed by a cell.

In certain aspects, a phenotypic property may be a physical characteristic of the cell such as cell size, internal composition of the cell (such as cell granularity) and cell autofluorescence. Cell size, cell granularity, and other physical characteristics of the cell that affect light scatter may be assessed by forward scatter (FSC), side scatter (SSC) or axial light loss (ALL), as well as any combination of two or more of these parameters. Cell autofluorescence may be assessed as fluorescence excitation spectrum that does not result mainly from a label in the cell (e.g., that results instead from endogenous molecules in the cell with fluorescent properties).

In certain aspects, a phenotypic property may be a biochemical characteristic of the cell such as expression (e.g., presence or amount) of cellular markers (e.g., cell surface markers, intracellular proteins, other molecules expressed by a specific cell type). Expression of a cellular marker may be assessed based on a signal provided by a label domain of a detectable label. A binding domain of the detectable label may specifically bind the cellular marker. In contrast to identifying expression of a cellular marker, identification of a physical characteristic of the cell may not require the use of a detectable label.

Aspects of the methods may further include sub-categorizing each of the first and second populations into sub-populations of cells based on a second and third phenotypic property using X detectable labels providing Y distinct signals, wherein X>Y, to identify sub-populations of cells in the cellular sample. In certain aspects, two detectable labels providing a similar or substantially identical signal may be distinguished based on the categorization of the primary populations. The distinguished detectible labels may be used to further identify (e.g., define, categorize, characterize) sub-populations of cells in the cellular sample.

In certain aspects, the cellular sample of the above described methods may be obtained from tissue, in vitro cell culture, etc. For isolation of cells from tissue, an appropriate solution may be used for dispersion or suspension. The solution may be a balanced salt solution, e.g., normal saline, PBS, Hank's balanced salt solution, etc., conveniently supplemented with fetal calf serum, human platelet lysate or other factors, in conjunction with an acceptable buffer at low concentration, such as from 5-25 mM. Convenient buffers include HEPES, phosphate buffers, lactate buffers, etc. The separated cells may be collected in any appropriate medium that maintains the viability of the cells. Various media are commercially available and may be used according to the nature of the cells, including dMEM, HBSS, dPBS, RPMI, Iscove's medium, etc., frequently supplemented with fetal calf serum or human platelet lysate. The cellular sample may include mammalian (e.g., human, murine) or non-mammalian cells.

The cellular sample may be contacted with detectable labels. A detectable label may include a binding domain and a label domain. The terms “specific binding,” “specifically binds,” and the like, refer to the preferential binding of a domain (e.g., one binding pair member to the other binding pair member of the same binding pair) relative to other molecules or moieties in a solution or reaction mixture. The binding domain may specifically bind (e.g., covalently or non-covalently) to a particular epitope or narrow range of epitopes within the cell. In certain aspects, the binding domain non-covalently binds to a target. In such instances, the binding domain association with the binding target may be characterized by a KD (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less.

A variety of different types of binding domains may be employed. Binding domains of interest include, but are not limited to, antibody binding agents, proteins, peptides, haptens, nucleic acids, etc. The term “antibody binding agent” as used herein includes polyclonal or monoclonal antibodies or binding fragments thereof that are sufficient to bind to an analyte of interest. The binding fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

The label domain may be detectable based on, for example, fluorescence emission maxima, fluorescence polarization, fluorescence lifetime, light scatter, mass, molecular mass, or combinations thereof. In certain aspects, the label domain may be a fluorophore (i.e., a fluorescent label, fluorescent dye, etc.). Fluorophores can be selected from any of the many dyes suitable for use in analytical applications (e.g., flow cytometry, imaging, etc.). A large number of dyes are commercially available from a variety of sources, such as, for example, Molecular Probes (Eugene, Oreg.) and Exciton (Dayton, Ohio). Examples of fluorophores that may be incorporated into the microparticles include, but are not limited to, 4-acetamido-4′-isothiocyanatostilbene-2,2′disulfonic acid; acridine and derivatives such as acridine, acridine orange, acrindine yellow, acridine red, and acridine isothiocyanate; 5-(2′-aminoethyl)aminonaphthalene-1-sulfonic acid (EDANS); 4-amino-N[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used. The fluorescent label may be distinguishable based on fluorescence emission maxima, and optionally further based on light scatter or extinction.

In other aspects, the label domain may be a metal isotope detectable by mass spectroscopy, such as by the time of flight mass spectrometer used in mass cytometry, e.g., as described in international patent application serial no. PCT/US2012/020950 published as WO/2010/097070, the disclosure of which is herein incorporated by reference.

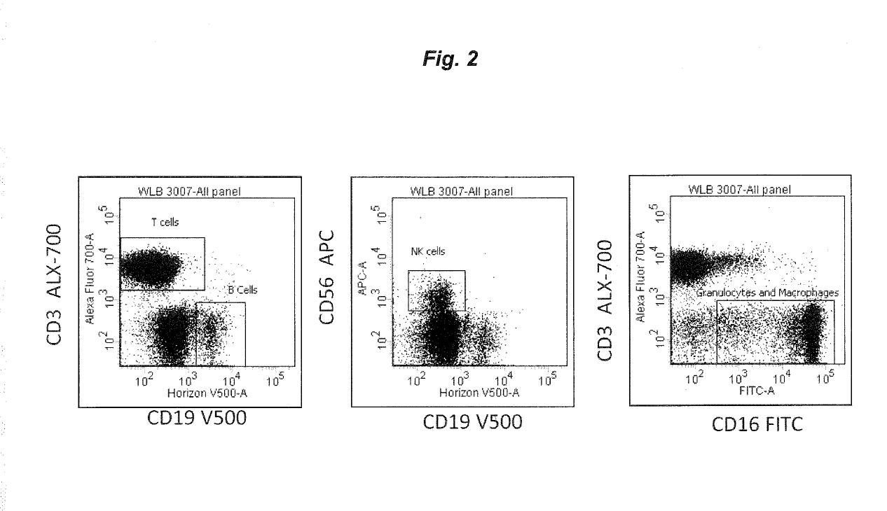

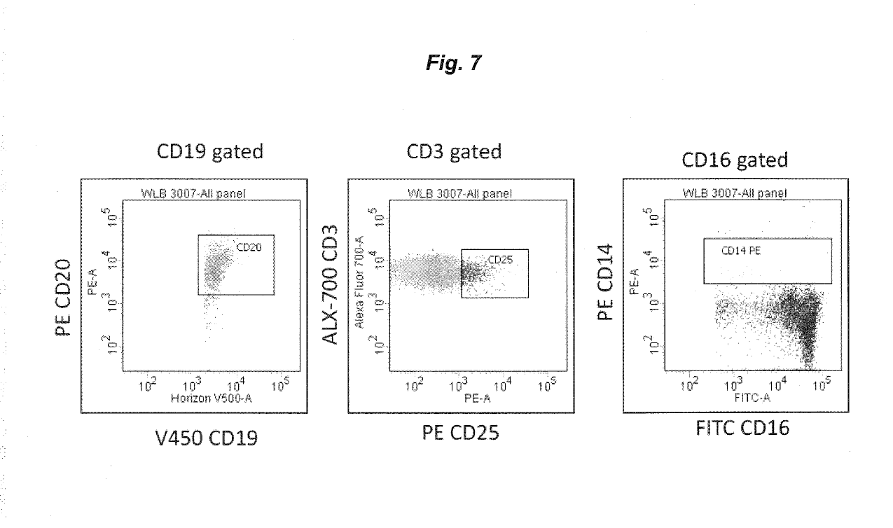

In certain aspects, the label domains of two or more detectible labels may provide a substantially identical signal. Specifically, the label domains may be identical as seen in FIG. 7, or may otherwise provide a similar signal, such as the overlapping fluorescence emission seen in FIG. 2.

The cellular sample may be contacted with the detectable label(s) at the same time or in succession. The sample may be contacted with a sufficient amount of the detectable labels and for a period of time sufficient to allow binding of detectable labels to their specific targets. For example, the sample may be contacted for between 5 minutes and several hours, such as between 30 minutes and 2 hours. The sample may be maintained and any convenient temperature, e.g., between freezing and room temperature during the contacting step. A washing step may then be performed, as desired, e.g., to remove any unbound detectable labels and other sample components. Washing may be performed using any convenient protocol, such as by combining the reaction mixture with a suitable wash buffer (e.g., PBS, HEPES) and separating the cells from the fluid. A given washing protocol may include one or more distinct washing steps, as desired. Following any washing protocol, the cells may be re-suspended in a suitable liquid (e.g., the washing buffer or another buffer). In certain aspects, a detectable label may specifically bind a cellular marker such as CD4, CD8, CD25, CD7, CD20, CD79b, CD10, CD79a, CD33, CD64, CD13, CD15, CD117, CD135, CD105, CD44, CD73, CD54, CD274, IL-6, and FoxP3.

In certain aspects, the method may further include fixing the cellular sample prior to and/or after labeling the cellular sample. The cells of the sample may be fixed through exposure to any of a number of cell fixing agents (i.e., fixation reagents), such as paraformaldehyde, glutaraldehyde, methanol, acetone, formalin, or any combination thereof. Other fixatives and fixation methods may be employed, as desired. Fixation time may vary, and in some instances ranges from 1 minute and 1 hour, such as 5 minutes and 30 minutes. The temperature at which fixation takes place may vary, and in some instances the temperature ranges from −30° C. to 30° C.

In some instances, the detectable labels may be quantified by flow cytometry. Flow cytometry is a methodology using multi-parameter data for identifying and distinguishing between different particles, such as cells or beads, that vary from one another (e.g., in terms of label, size, granularity, etc.) in a fluid medium. In flow cytometrically analyzing the particles (e.g., the cells prepared as described above), a liquid medium including the particles is first introduced into the flow path of the flow cytometer. When in the flow path, the particles are passed substantially one at a time through one or more sensing regions, where each of the particles is exposed individually to a source of monochromatic light and measurements of light scatter parameters and/or fluorescent emissions as desired (e.g., two or more light scatter parameters and measurements of one or more fluorescent emissions) are separately recorded for each particle.

In series with a sensing region, a detector module that includes one or more detectors, e.g., light collectors, such as photomultiplier tubes (or “PMT”), is used to record light that passes through each particle (generally referred to as forward light scatter), light that is reflected orthogonal to the direction of the flow of the particles through the sensing region (generally referred to as orthogonal or side light scatter), light loss along the axis of irradiation (generally referred to as axial light loss), and fluorescent light emitted from the particles as the particle passes through the sensing region and is illuminated by the energy source. Forward light scatter (or FSC), orthogonal light scatter (SSC), axial light loss (ALL) and fluorescence emissions include separate parameters for each particle (i.e., each “event”).

More specifically, in a flow cytometer, the particles are passed, in suspension, substantially one at a time in a flow path through one or more sensing regions where in each region each particle is illuminated by an energy source. The energy source may include an illuminator that emits light of a single wavelength, such as that provided by a laser (e.g., He/Ne or argon) or a mercury arc lamp with appropriate filters.

Accordingly, in flow cytometrically assaying the particles, the particles which may include different amounts of each detectable label are detected by exposing the particles to excitation light and measuring the fluorescence of each particle in one or more detection channels, as desired. The excitation light may be from one or more light sources and may be either narrow or broadband. Examples of excitation light sources include lasers, light emitting diodes, and arc lamps. Fluorescence emitted in detection channels used to identify the particles and binding complexes associated therewith may be measured following excitation with a single light source, or may be measured separately following excitation with distinct light sources. If separate excitation light sources are used to excite the detectable labels, the labels may be selected such that all the labels are excitable by each of the excitation light sources used.

Flow cytometers may further include data acquisition, analysis and recording means, such as a computer, wherein multiple data channels record data from each detector for the light scatter and fluorescence emitted by each particle as it passes through the sensing region. The purpose of the analysis system is to classify and count particles wherein each particle presents itself as a set of digitized parameter values.

In flow cytometrically assaying particles in methods of the invention, the flow cytometer may be set to trigger on a selected parameter in order to distinguish the particles of interest from background and noise. “Trigger” refers to a preset threshold for detection of a parameter. It is typically used as a means for detecting passage of particle through the laser beam. Detection of an “event” (e.g., a particle such as a bead or cell) that exceeds the preset threshold for the selected parameter triggers acquisition of light scatter and fluorescence data for the particle. Data is not acquired for particles or other components in the medium being assayed which cause a response below the threshold. The trigger parameter may be the detection of forward scattered light caused by passage of a particle through the light beam. The flow cytometer then detects and collects the light scatter and fluorescence data for particle. The flow cytometer may thereby produce a data set (e.g., signal data such as FSC, SSC, fluorescence emission, etc., from each event).

In certain embodiments, detectable labels may be distinguished based on fluorescence emission (e.g., fluorescence emission maxima). For example, fluorescence compensation between two or more detectable labels with spectral overlap may be employed to distinguish the signal (e.g., fluorescence emission) resulting from each of the detectable labels. Two or more detectable labels may also be distinguished based on light scattering, fluorescence lifetime, excitation spectra, or combinations thereof.

A particular population of interest may be categorized (e.g., “gated”) based on the data set collected for the entire sample. To select an appropriate gate, the data set is plotted so as to obtain the best separation of populations possible. This procedure is typically done by plotting forward light scatter (FSC) vs. side (i.e., orthogonal) light scatter (SSC) on a two dimensional dot plot (e.g., a linear or log scale scatter plot). Particles (e.g. cells, beads, also referred to as “events”) may be gated into separate populations based on differences in FSC and/or SSC intensity. For example, populations may differ from one another in FSC and/or SSC intensity by two-fold or more, five-fold or more, or ten-fold or more. The flow cytometer operator then selects the desired population of particles (i.e., those cells within the gate) and excludes particles that are not within the gate. Where desired, the operator may select the gate by drawing a line around the desired subpopulation using a cursor on a computer screen. Only those particles within the gate are then further analyzed by plotting (e.g., on a linear or log scale) the other parameters for these particles, such as fluorescence. Gating based on fluorescence may then be used to further categorize populations of cells. Particles may be gated into separate populations based on fluorescence emission, a lack of fluorescence emission, or differences in fluorescence (e.g. fluorescence emission maxima). In some examples an average (e.g. mean, median) fluorescence by two-fold or more, five-fold or more, or ten-fold or more.

In certain aspects, primary cell populations, also referred to herein as cell types, may be categorized (e.g., gated) based on phenotypic properties. The term “phenotypic property” is used broadly to refer to any observable physical or biochemical characteristics of an cell.

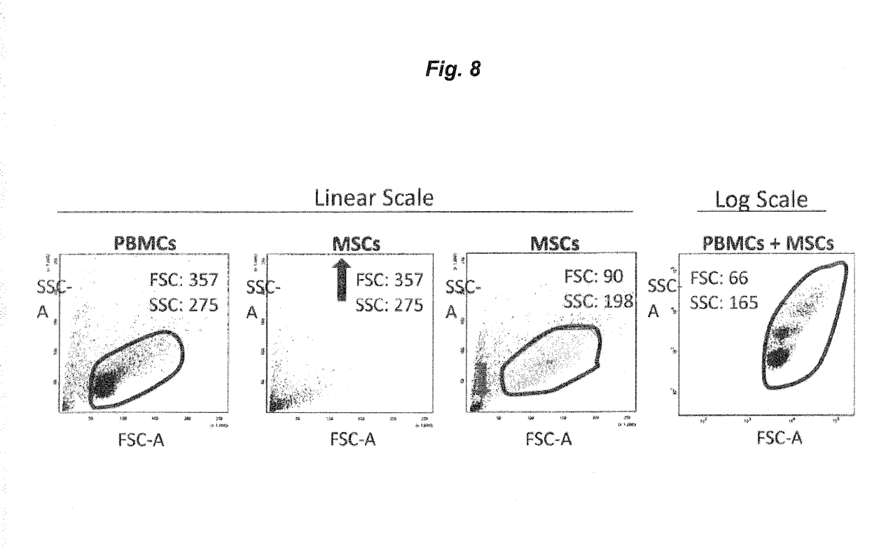

Physical characteristics of a cell may include cell size, internal composition of the cell (such as granularity), cell autofluorescence, etc. The size and internal composition of the cell affects light scatter properties of the cell. Light scatter may therefore be measured to distinguish cell populations based on phenotypic properties such as size and internal composition. For example, cell size may be assessed by forward scatter (FSC), side scatter (SSC) or axial light loss (ALL), as well as any combination of two or more of these parameters. Internal composition (e.g., cell granularity) may be identified by SSC. For cell populations exhibiting significant difference in FSC or SSC, use of log scale may be useful in categorizing the parent populations, as seen in FIG. 8. Cell autofluorescence may be assessed by fluorescence excitation, fluorescence emission spectra, or a combination thereof. As cell autofluorescence tends to be more pronounced at lower wavelengths within the ultraviolet to infrared range, cell autofluorescence may be identified based on fluorescence excitation and/or emission spectra of less than 700 nm, less than 650 nm, less than 600 nm, less than 550 nm, less than 500 nm, less than 450 nm or less than 400 nm. For cell populations exhibiting significantly different levels of auto-fluorescence, voltage settings may be optimized for the cell population exhibiting higher auto-fluorescence.

Biochemical characteristics of a cell include expression (e.g., presence or amount) of cellular markers such as any cell surface markers, intracellular proteins, or other molecules expressed by a specific cell type. Expression of a cellular marker may be assessed based on a signal provided by a label domain of a detectable label. A binding domain of the detectable label may specifically bind the cellular marker.

One aspect of this invention is the utilization of multicolor flow cytometry combined with the known rules for expression on cells, such as specific types of differentiated blood cells. The use of primary markers (e.g., surface markers, intracellular proteins, other molecules expressed by a specific cell type) that may distinguish primary cell populations (e.g., such as the use of CD3 for all T cell subsets and CD19 for B cells) may be used to gate, classify, or otherwise categorize signals from secondary markers. Utilizing this concept, combined with identifying specific subsets of cells (secondary populations or sub-populations) under each primary marker, the same fluorescent detectors may be used to detect label domains (e.g., fluorescent labels) which have been conjugated to two or more different binding domains (e.g., cell surface receptor specific antibodies).

In one example, PE conjugated anti-CD4 may be distinguished under the CY5 conjugated T cell specific primary marker from PE conjugated anti-B220 which may be distinguished under the FITC conjugated B cell specific primary marker. Thus a user may be able to distinguish all four populations (e.g., Total T cells, Total B cell, CD4 Positive T cells and B220 Positive B cells) using only 3 colors, since the sub-populations for the primary populations (T and B cells) use the same fluorescent dye on their respective antibodies but are distinguished by the fluorescent signal of the primary marker. As discussed above, categorization of primary populations may be based on other phenotypic properties such as autofluorescence, cell size and/or composition as measured by FSC and/or SSC, instead of or in addition to primary markers, e.g., as seen in FIGS. 8-11.

Conventional flow cytometry restricts the use of a single dye/fluorophore to a given specificity, and thus a given multicolor experiment may only detect a set of markers for which an equal number of dyes may be used. This may restrict the experiment to, for example, the number of detectors in flow cytometric system, or the number of dyes available for a particular system. Aspects of the present invention beneficially allow for the expansion of multi-color flow cytometry analysis by providing a plurality of specificities to be conjugated with a single detectable signal such as from a single dye or fluorophore. In some embodiments the use of a primary marker may be used to create a unique gate for detection of subset cell types within the primary marker population.

This approach is illustrated in Table 1 (below) and where it can be seen that by adding minimally one extra cell type primary marker to the panel using only one additional required color, two, three, four, five or more additional markers may be detected depending on the original number of detectable signals utilized in the panel.

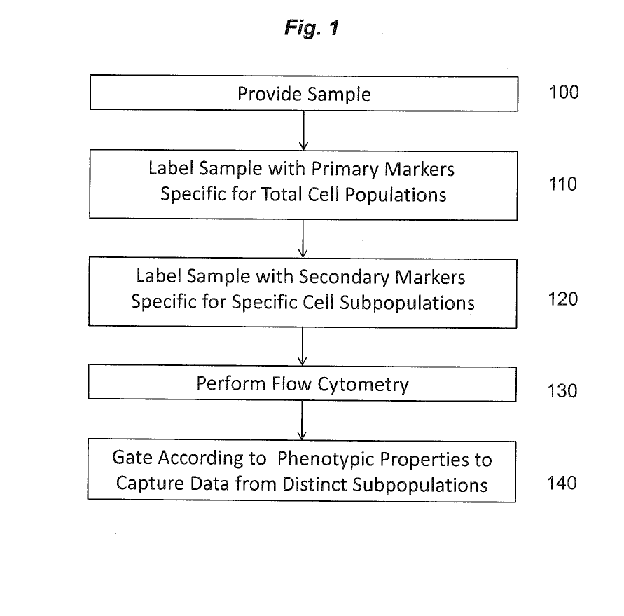

In general as depicted in FIG. 1, a sample may be provided and labeled with antibodies to generate n number of distinguishable signals from conjugated antibodies for markers P that are specific for a general cell type or particle type, and then labeled antibodies to provide m number of distinguishable signals from secondary makers S that are sub-populations of the cells defined by the primary markers. The total number of signals that may be observed is Y=n+m. The signals may be detected by performing a flow cytometry experiment to obtain a data set. Appropriate gating of the data set to isolate the signal from the primary cell types provides a method to capture data from the distinct sup-populations. Thus while the number of observed signals may be Y=n+m, the number of detectable markers X that may be detected is X=n(m+1). In some aspects of the invention, where there are two primary markers generating two distinguishable signals, the total number of detectable markers X will exceed the number of distinguishable detectable signals Y required for the single experiment. In some aspects, the number of secondary markers S will exceed the number of distinguishable signals from the secondary markers m. The signals may be generated by fluorescent dyes such as FITC, PE, V450, AmCyan, APC, PE-Cy™ 7, PerCP-Cy™ 5.5, V500-C, BV605 BV421, any of the fluorophores previously described for the label domain, etc. and be distinguished by any means such as the appropriate use of band pass filters.

TABLE 1

| | Cell TYPE | | | | |
|---|---|---|---|---|---|
| | T cell | B cell | NK | Granulocyte | Cell Type 5 |
| | Primary Labels | | | | |
| | CD3 (P₁)- | CD19 (P₂)- | CD56 (P₃)- | CD16 (P₄) | (Pₙ) |
| Secondary Labels | Color 1 | color-2 | color-3 | color-4 | color-n |
| Color-n + 1 | T cell sub | B cell sub 1b | NK cell sub 1c | Mono sub 1d | Cell Type 5 sub 1e |
| | 1a | (S₂) | (S₃) | (S₄) | (S₅) |
| | (S₁) | | | | |
| Color-n + 2 | T cell sub | B cell sub | NK cell sub | Mono sub 2d | Cell Type 5 sub 2e |
| | 2a (S₆) | 2b(S₇) | 2c(S₈) | (S₉) | (S₁₀) |
| Color-n + 3 | T cell sub | B cell sub 3b | NK cell sub 3c | Mono sub 3d | Cell Type 5-sub 3e |
| | 3a | (S₁₂) | (S₁₃) | (S₁₄) | (S₁₅) |
| | (S₁₁) | | | | |
| Color-m | T cell sub | B cell sub 4b | NK cell sub 4c | Mono sub 4d | Cell Type 5-sub 4e |
| | 4a | (S₁₇) | (S₁₈) | (S₁₉) | (S_{X}) |
| | (S₁₆) | | | | |

Additional advantages of this approach will be to expand the utility of every type of flow cytometer in order to generate a higher complexity multicolor experiment. As seen in Table 1, starting with a simple 5 color experiment with markers for 5 primary cell types and adding one more color, a scientist may achieve the detection of 10 different species in a single experiment as compared to a 6 color experiment yielding 6 specificities. Likewise a 10 color experiment would not be restricted to 10 specificities, but could report out 30 specificities.

In certain aspects, the number of cell type markers P and therefore total number of colors required to identify n cell types may be reduced by detecting a cell type based on phenotypic properties other than surface marker expression. Such phenotypic properties may include cell size and/or internal composition (e.g., as measured by FSC, SSC, ALL, etc.), autofluorescence or a combination thereof, as discussed previously. Further, a cell type may alternatively or additionally be categorized or “gated” based on a lack of expression of other cell type markers. In certain aspects, primary labels may be used in combination with detection of phenotypic properties other than surface marker expression, e.g., to enhance accuracy of the categorization of cell types. Conventionally, scientists using multicolor approaches to detect cells and subsets of cells, for example T cells and T cell subsets (and/or intracellular proteins) simply use additional or separate tubes to detect the cellularity of a population. The categorization based on phenotypic properties described herein may allow the scientist to generate the same data within a single reaction (tube) or sample and thus allow a more detailed landscape visualization of cell types, or work with particularly small sample sizes. This approach may allow all scientists to get a deeper insight into a heterogeneous population by incorporating a single approach to visualizing many different cell types and subsets of cells in a given population. In one aspect this approach may allow a complete categorization and characterization of an entire heterogeneous population of cells within a single multicolor flow cytometry test. Using the approach of multiple primary cells and antibodies for any cell type combination may be utilized such as a Pluripotent Stem Cell, mesenchymal stem cell (MSC), embryonic stem cell, hematopoietic stem cell, T cell, B cell, NK cell, Plasmacytoid Dendritic cell, Megakaryocyte, Endothelial cell, Neutrophil, MDSC, eosinophil, epithelial cell, mast cell, myeloid dendritic cell, Macrophage, Basophil, eosinophil. Subsets of each cell type may be detected at the same time with antibodies specific for individual sub-populations, but conjugated to overlapping dye sets. Cell sub populations that may be simultaneously detected by methods of this invention may include CD4, CD8, CD25, CD7, FoxP3 subtypes in T-cells and/or CD20, cd79b, CD10, CD79a subtypes in B-cells, and/or CD33, CD64, CD13, CD15 subtypes in Monocytes, CD117, CD135, CD105, CD44 in Stem cells, and/or CD73, CD54, CD274, IL-6 high/low populations of MSCs. In some embodiments the methods of this invention may provide for improved analysis of very small sample sizes, such as on the order of 5,000 or 1000 or fewer cells. The improved analysis may include the detection of 10 or 20 or 30 or more cell surface markers in a single experiment. In some embodiments this approach may be integrated into the front end of drug development multicolor flow application to screen the impact of drugs on the entire hematopoietic blood cell system, for on and off-target effects of a drug.

In certain aspects, the method may include assessing expression of cellular markers in specific cell populations (e.g., primary populations, sub-populations thereof) in a cellular sample. The amount (e.g., expression) of the cellular markers may be assessed to further categorize and/or characterize cell populations. The cellular sample may be treated prior to labeling to facilitate detection of cellular markers that are intracellular (e.g., cytokines that have not been secreted, transcription factors, other intracellular proteins, RNA, etc.).

In certain aspects, the method may involve treating the cellular sample with a protein transport inhibitor. Examples of protein transport inhibitors include Brefeldin A and Monensin, although other protein transport inhibitors may also be employed, as desired. Pretreating the MSC population with a protein transport inhibitor allows for the accumulation of normally secreted proteins (such as IL-6 and other cytokines) which may otherwise be difficult to detect. The cellular sample may be pretreated with the protein transport inhibitor for an amount of time sufficient to accumulate normally secreted proteins, such as from 5 minutes to 1 day, 30 minutes to 6 hours, or 1 hour to 2 hours.

Alternatively or in addition to being treated with a protein transport inhibitor, the sample may be treated with a permeabilization agent. Permeabilization may allow detectable labels which are specific for intracellular proteins, transcription factors and/or cytokines to enter the cell. Permeabilization may take place before, after, or at the same time as the fixation previously described. The cells of the sample may be permeabilized through exposure to any of a number of cell permeabilizing agents, such as methanol, acetone or a detergent (e.g., triton, NP-40, saponin, tween 20, digitonin, leucoperm, etc.), or a combination thereof. Permeabilization time may vary, and in some instances ranges from 1 minute to 1 hour, such as from 5 minutes to 30 minutes. The temperature at which permeabilization takes place may vary, and in some instances the temperature may range from 0° C. to 50° C.

In certain aspects, the cellular sample may include a co-culture. The co-culture may include at least two distinct populations of cells. The co-culture may be maintained in appropriate growth medium in suspension or plated, for a period of time that may vary based on the application. A cellular sample of the co-culture may be categorized into primary populations based on differences in phenotypic properties, as described above. Optionally further, sub-populations may be categorized according to any of the embodiments previously described. Cell populations (primary and/or sub-populations) may also be characterized for expression of cellular markers, as previously described.

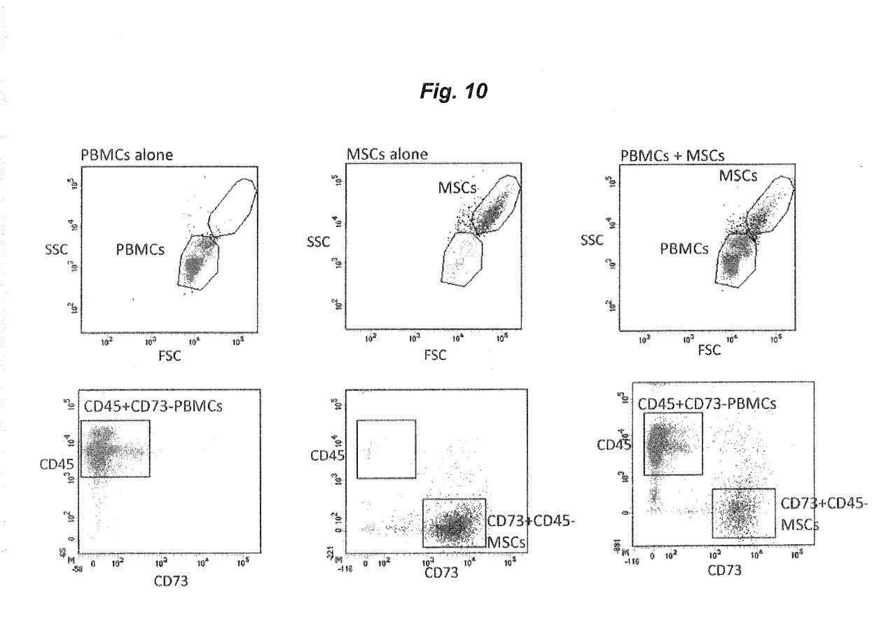

The co-culture may include adult stem cells (e.g., MSCs, hematopoietic stem cells, endothelial progenitor cells, etc.), pluripotent stem cells (embryonic stem cells, induced pluripotent stem cells, etc.) and mature cells (e.g., PBMCs, purified monocytes, lymphocytes, dendritic and NK cells, endothelial cells, cardiomyocytes, osteocytes, chondrocytes, adipocytes, etc.). Adult stem cells and mature cells may be categorized into separate cell populations based on their phenotypic properties (e.g., as seen in FIG. 10).

In some aspects, the co-culture may include MSCs and PBMCs. MSCs (aka mesenchymal stem cells, medicinal stem cells, medicinal stromal cells, multipotent stromal cells) may be plastic adherent and are capable of differentiation into multiple mesenchymal lineages, such as to osteoblasts, adipocytes, myoblasts and chondroblasts. Human MSCs may be positive for surface markers CD73, CD90, and CD 105, and negative for surface markers CD34, CD45, CD14, CD11 b, and CD19. In addition, other markers such as CD271, COX2, IDO, CD274, CD44, CD166, STRO-1 may be useful in identifying and/or characterizing human MSCs or subsets thereof. A thorough review of MSC populations and MSC surface markers can be found Hass R. et al. 2011, Cell Commun Signal. 14; 9:12., the disclosure of which is incorporated herein by reference. It should be noted that the criteria for identifying MSCs is not definitive as MSCs may differ in morphology, expression of surface markers, and/or immunomodulatory potential based on tissues of origin, culture conditions, species, or combinations thereof.

In certain aspects, MSCs may be produced by first obtaining cells (including MSCs and/or stem cells (SCs)) from mammalian tissue (e.g. as described previously). The mammalian tissue may be of a human, non-human primate, murine, or another suitable mammal. The tissue may be bone marrow, adipose tissue, peripheral blood, or another tissue suitable for producing MSCs.

The obtained cells may then be cultured under conditions suitable for MSC production and/or expansion. The culture conditions may include one or more passages and in some instances ten or fewer passages. The culture conditions may include one or more factors for maintaining multipotency in cells. Examples of such factors include fetal bovine serum (FBS), human platelet lysate, vectors for transfecting genes for inducing/maintaining pluripotency, etc. The MSC population may be frozen (e.g., in 5% or greater DMSO and at liquid nitrogen temperatures) prior to use, as desired.

MSCs as described above can be propagated continuously in culture, using culture conditions that promote proliferation without promoting differentiation, as desired. The cells can be maintained in medium, e.g., DMEM, RPMI, etc., in the presence of fetal bovine serum or serum-free replacement without differentiation. The cells may be passaged at 75 to 95% confluence, using a protease, e.g., trypsin, collagenase, etc. Due to the multipotency of MSCs, and despite their relative rarity in their tissue of origin (often a fraction of a percent), MSCs propagated in culture may be enriched to levels suitable for clinical applications.

In certain aspects, a substantially pure population of MSCs may be obtained by enriching for MSCs or SCs that are precursors to MSCs, wherein any convenient protocol for doing so may be employed. For example, beads conjugated to antibodies (or another binding molecule) that specifically bind to non-MSC surface markers may be used to deplete non-MSC cells. Beads conjugated to antibodies specific for MSC surface markers may be used to separate MSCs from other cells. In another example, a gating strategy similar to that illustrated in FIG. 10 may be employed on a fluorescence activated cell sorter (FACS) instrument to purify the MSC population.

The tissue of origin and culture conditions can lead to MSC populations with different characteristics (such as surface marker expression) and immunomodulatory potential. As such, different batches of MSCs may exhibit different therapeutic efficacy. Levels of cellular markers expressed by MSCs may correlate with immunomodulatory potential.

The immunomodulatory potential of the MSC population may be an ability of the MSC population to suppress proliferation and/or activation of certain immune cells, such as T-cells, B-cells, NK-cells, or combinations thereof. Immunomodulatory potential of the MSC population may also include the ability of the MSCs in the population to modulate immune cell development (e.g., induce T-cell differentiation into regulatory T-cells, prevent monocyte differentiation into dendritic cell, etc.).

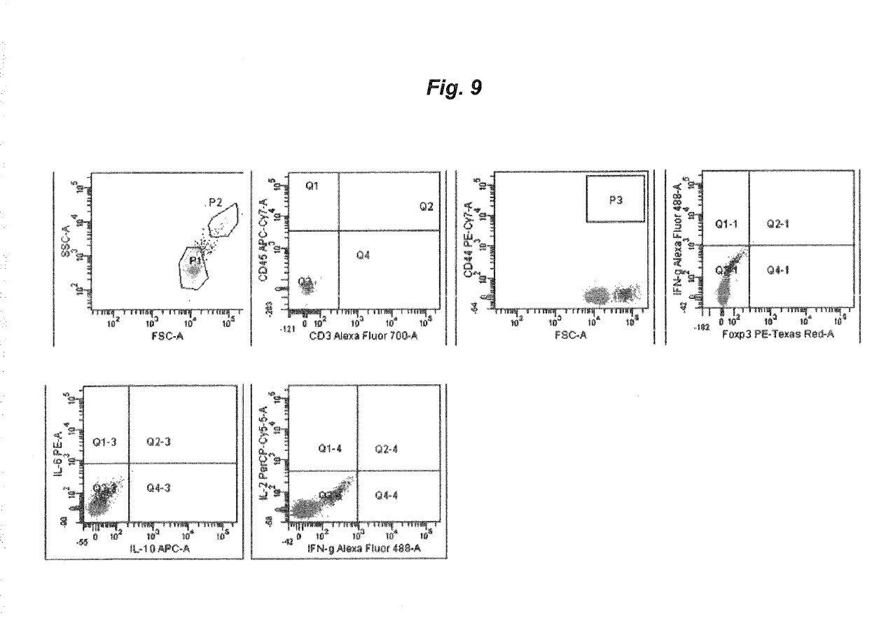

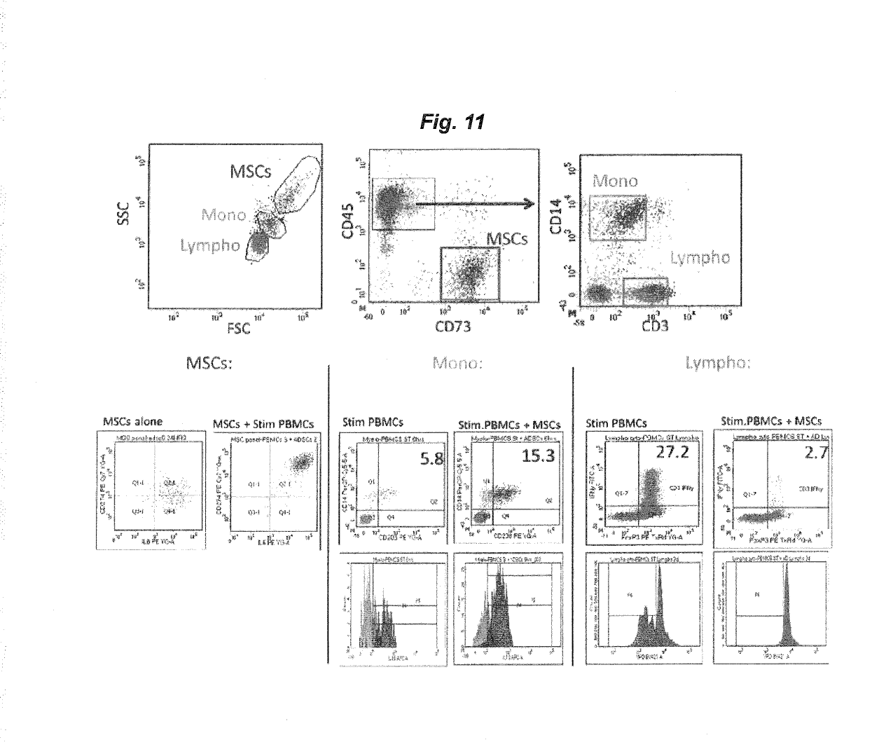

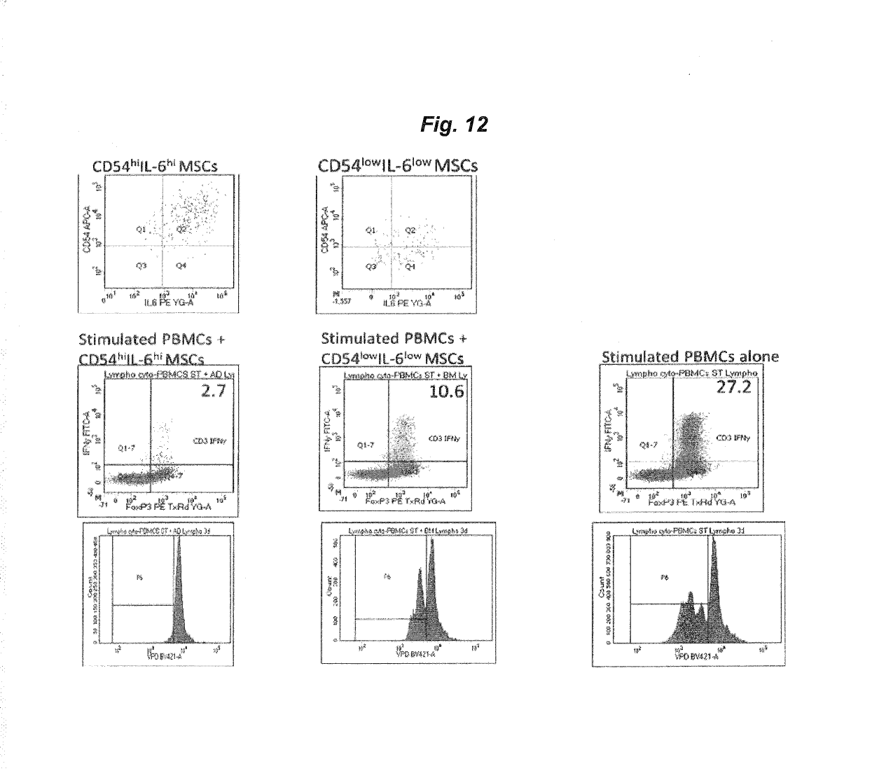

In certain aspects, a co-culture of MSCs and PBMC may be discriminated (e.g. categorized, gated) in a log scale. Voltage settings may be optimized based on MSCs showing higher autofluorescence as seen in FIGS. 8 and 9. PBMCs may be discriminated based on size and exclusive expression of CD45 while MSCs can be discriminated based on size and expression of CD73 and lack of CD45 as seen in FIG. 10. Multicolor flow cytometric characterization of the MSC population categorized by the above methods allows for the determination that, for example, 1) IL-6 and CD274 expression by MSCs is upregulated in the presence of stimulated PBMCs; 2) IL-10-expressing CD14+CD206+ macrophages are up-pregulated in the presence of MSCs; and 3) Stimulated PBMC proliferation and IFN-γ expression are inhibited in the presence of MSCs (as seen in FIG. 11). Comparing functionally different MSCs using the aspects of the methods described herein allows the signature of immunosuppressive MSCs to be defined. Data generated using aspects of this invention suggest that adipose-derived MSCs expressing high levels of CD 54 and IL-6 are better at inhibiting T-cell activation than bone marrow-derived MSCs expressing low levels of CD54 and IL-6, as seen in FIG. 12. This signature may be used to screen MSCs prior to use in clinical settings to predict their immunosuppressive efficacy.

Methods of this invention may be performed manually on data sets generated by a flow cytometer. In some embodiments a computer readable code may be used to automatically distinguish between cell type sub-populations labeled with the same fluorescent dye based on the gating parameters of primary cell types. The primary cell types may be gated manually or via the utilization of computer readable code. The following non-limiting examples further illustrate the present invention.

### Devices and Systems

Aspects of the invention further include systems for use in practicing the subject methods. Systems of the invention may include a flow cytometry system configured to assay particles (e.g., beads, cells such as MSCs, etc.) by measuring signals such as FSC, SSC, ALL, fluorescence emission (e.g., as emission maxima), mass, molecular mass, etc. Steps of the methods described in the previous sections may be performed by the flow cytometry system. Flow cytometers of interest include, but are not limited, to those devices described in U.S. Pat. Nos. 4,704,891; 4,727,029; 4,745,285; 4,867,908; 5,342,790; 5,620,842; 5,627,037; 5,701,012; 5,895,922; 6,287,791; 7,787,197; 8,140,300; and 8,528,427; the disclosures of which are herein incorporated by reference.

In some instances, the flow cytometer includes: a flow channel; a detector module that includes a first detector configured to receive a first signal from the assay region of the flow channel and a second detector configured to receive a second signal from the assay region of the flow channel. The flow cytometer may optionally further include at least a first light source configured to direct light to an assay region of the flow channel (where in some instances the cytometer includes two or more light sources). Optionally further, the flow cytometer may include one or more additional detectors and/or light sources for the detection of one or more additional signals. The one or more additional signals may be produced by one or more additional detectable labels.

The flow cytometer may be configured to produce a data set. The data set may include signal data (e.g., fluorescence excitation and/or emission spectra, fluorescence intensity, fluorescence emission maxima, FSC, SSC, ALL or combinations thereof) for each event in the data set.

The flow cytometry system may also include a “data processing unit”, e.g., any hardware and/or software combination that will perform the functions required of it. For example, any data processing unit herein may be a programmable digital microprocessor such as available in the form of an electronic controller, mainframe, server or personal computer (desktop or portable). Where the data processing unit is programmable, suitable programming can be communicated from a remote location to the data processing unit, or previously saved in a computer program product (such as a portable or fixed computer readable storage medium, whether magnetic, optical or solid state device based).

The flow cytometry system may further include a “memory” that is capable of storing information such that it is accessible and retrievable at a later date by a computer. Any convenient data storage structure may be chosen, based on the means used to access the stored information. In certain aspects, the information may be stored in a “permanent memory” (i.e. memory that is not erased by termination of the electrical supply to a computer or processor) or “non-permanent memory”. Computer hard-drive, CD-ROM, floppy disk, portable flash drive and DVD are all examples of permanent memory. Random Access Memory (RAM) is an example of non-permanent memory. A file in permanent memory may be editable and re-writable.

The memory may store a “module” for execution by the data processing unit, wherein the module is configured to transform the data set from a number transform the data set from a number (X) of signal sets to a number (Y) of marker density sets, wherein Y>X. The marker density sets may include marker expression data (e.g., levels and/or amounts of cellular markers, signals from detectible labels corresponding to cellular markers, etc.) for each cell event in the data set or in a population thereof. The module may be configured to transform the data set based on a categorization of events (e.g. cell events) in the signal set. For example, the same fluorescent signal obtained from two cell events categorized into separate populations may be provided by different detectable labels specific for different cell marker. The module may be configured to distinguish detectable labels (e.g., detectable labels providing a substantially identical signal) based on the categorization.

In certain aspects, the module may be configured to categorize the cell events prior to transforming the data set. Further, the module may be configured to categorize the cell events based on measurements of FSC, SSC, ALL, fluorescence emission or combinations thereof. In other aspects, the cell events may be categorized by an operator (i.e., manually) as described previously.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

In some instances, the systems may further include a cellular sample (e.g., loaded on the flow channel), as prepared according to any of the aspects of the subject methods described above. In certain aspects, the flow cytometer may be a fluorescence activated cell sorter (FACS) instrument or a mass cytometer.

### Utility

The methods of categorizing primary populations based on phenotypic properties (e.g., such as cell size, cell internal composition, cell autofluorescence, cell marker expression, and combinations thereof) have a number of useful applications described below.

Aspects of the methods described herein include categorization of primary populations based on phenotypic properties to further expand the number of specificities simultaneously analyzed. Specifically, methods herein provide a way to increase the number of cell markers assayed by distinguishing similar or identical signals from different detectable labels based on the categorization of primary populations and/or sub-populations thereof. Furthermore, in certain aspects, the number of detectable labels used to categorize the primary populations may be reduced by categorizing one or more primary populations based on phenotypic properties other than cell marker expression.

Certain aspects of the methods allow cell populations to be categorized flow cytometrically. As such, cells populations do not need to be cultured in separate wells (transwell system, culture with conditioned medium) or physically separated by immunoselection in order to determine which cell component (e.g., population) expresses a molecule of interest. Each cell population may be separately interrogated for the expression of molecules (e.g., surface markers, cytokines, transcription factors, etc.) of interest. By preserving the cell-to-cell contact between different cell types, aspects of this invention enable a deep and comprehensive characterization of co-culture cross-talk.

Certain aspects of the methods include the ability to define the mechanism(s) underlying the immunosuppressive ability of mesenchymal stromal cells (aka mesenchymal stem cells, medicinal stem cells, medicinal stromal cells, multipotent stromal cells, MSCs). In addition, aspects of the methods find use as standardized analytical tools to study the ability of MSCs to interact and functionally alter immune cells.

### Kits

In yet another aspect, the present invention provides kits for practicing the subject methods, e.g., as described above. The subject kits may include a first detectable label that specifically binds to a first cellular marker and a second detectable label that specifically binds to a second cellular marker. The first and second detectable labels may provide a substantially identical signal. A detectable label may include a label domain and a binding domain specific for a cellular marker, as described in the previous section. The binding domain of the first detectable label may be different than the binding domain of the second detectable label. For example, the binding domain of the first detectable label may specifically bind a cellular marker that the binding domain of the second detectable label cannot specifically bind to.

As described in the above sections, examples of cellular markers include cell surface markers, intracellular proteins (e.g. transcription factors), cytokines that have not been secreted, and the like. In certain embodiments, a cellular marker may be CD4, CD8, CD25, CD7, CD20, CD79b, CD10, CD79a, CD33, CD64, CD13, CD15, CD117, CD135, CD105, CD44, CD73, CD54, CD274, IL-6, and FoxP3.

In addition, the kit may include one or more additional detectable labels that specifically bind additional cellular markers. Detectable labels may be provided in separate containers or mixed in the same container.

The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The kit may further include reagents for performing a flow cytometric assay. Examples of said reagents include buffers for at least one of reconstitution and dilution of the first and second detectable molecules, buffers for contacting a cell sample with one or both of the first and second detectable molecules, wash buffers, control cells, control beads, fluorescent beads for flow cytometer calibration and combinations thereof.

The detectable labels and/or reagents described above may be provided in liquid or dry (e.g., lyophilized) form. Any of the above components (detectable labels and/or reagents) may be present in separate containers (e.g., separate tubes, bottles, or wells in a multi-well strip or plate). In addition, one or more components may be combined into a single container, e.g., a glass or plastic vial, tube or bottle.

In certain aspects, the kit may include one or more standardized controls. The standardized controls may be control particles such as control beads or control cells.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the internet to access the information at a removed site.

The following examples are offered by way of illustration and not by way of limitation.

### EXPERIMENTAL

The following experiment (FIGS. 1-7) was performed to provide an illustration of methods of this invention. White blood cells were prepared and stained according to standard protocols in which markers for various cell types and sub-populations were labeled in a single tube or in separate tubes with antibodies conjugated to fluorescent dyes as shown in Table 2 (below). Blood was collected from healthy donors using sodium heparin vacutainer tubes (BD 367874). Peripheral blood mononuclear cells (PBMC) were isolated using Ficoll pague plus (GE healthcare 17-1440-02). Isolated PBMC were washed with FACS buffer (1×PBS containing 2% FCS+0.09% Sodium Azide). Approximately 1×10⁶ cells were aliquoted into a 10×75 mm test tube then pelleted by centrifugation at 1400 rpm for 5 minutes. Supernatants were decanted then the cells were stained by adding optimal concentrations of fluorescent antibodies to each tube. In the case of the ‘all panel’ experiment, cells were stained with all of the labeled antibodies in the ‘all panel’ sample by adding optimal concentrations of fluorescent antibodies to a single tube. Cells were then incubated for 20-25 minutes on ice then washed by adding 1 ml of FACS buffer to each tube. Cells were pelleted by centrifugation, supernatants were decanted. Cells were re-suspended in 0.5 ml of FACS buffer then acquired using a BD Fortessa flow cytometer.

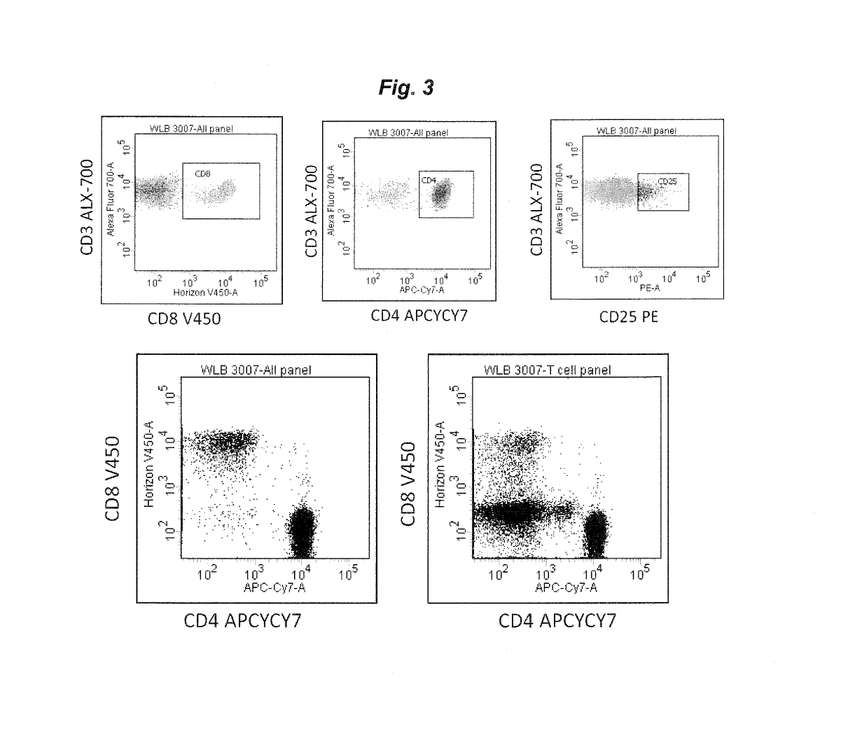

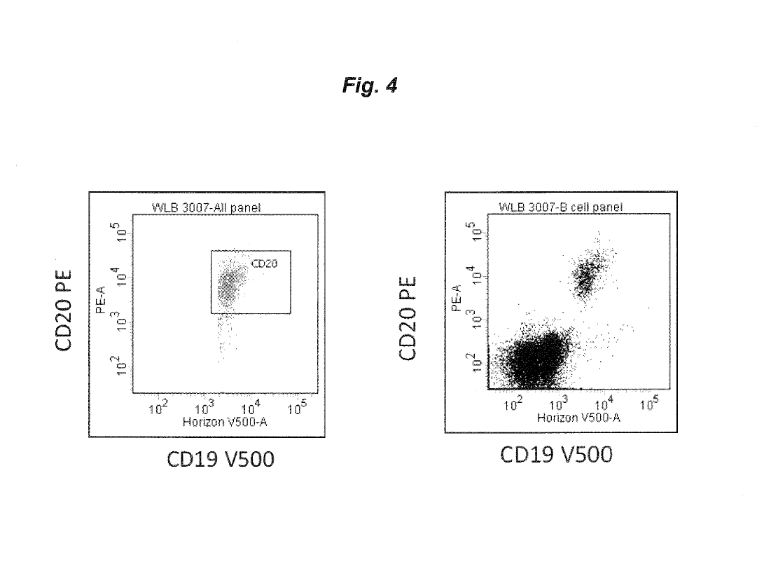

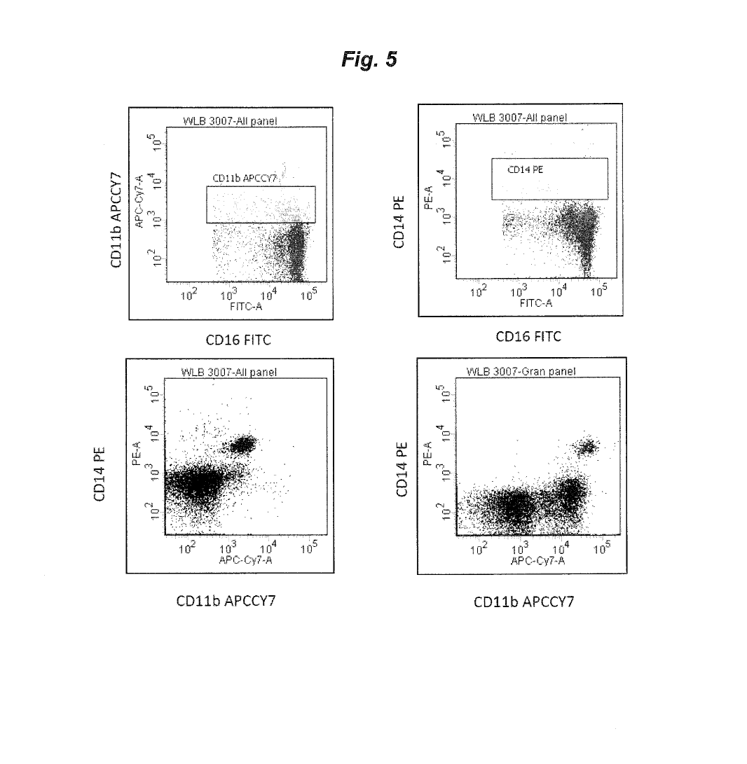

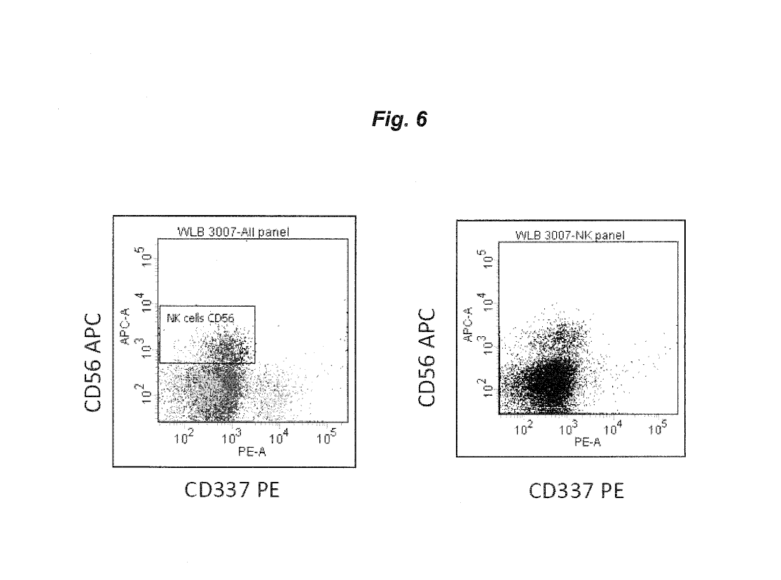

Primary and secondary cell surface markers were labeled a single tube in an ‘all panel’ experiment and additionally in four single cell type experiments were performed that included the labeling of at least one primary cell type and one secondary labeling of a sub-population of that cell type. Primary markers for T-cells, B-cells, NK cells and Granulocytes were labeled with antibodies conjugated to distinguishable fluorophores and flow cytometrically analyzed using a BD Fortessa™ flow cytometer. Density plots shown in FIG. 2 indicated how each of the primary cell types may be distinguished and gated based on the colorimetric properties of the conjugated antibody. Once the gate is established, particular secondary markers for sub-populations may be distinguished despite overlapping signal from the conjugated antibodies of the secondary markers. FIG. 3 shows density plots derived from the T-cell gate (CD3) of the ‘all panel’ experiment indicating that three subpopulations (CD6, CD4, and CD25) may be distinguished within that gate and that the distribution pattern in the ‘all panel’ experiment (bottom left) is similar to the distribution pattern derived from a T-cell only panel (bottom right). FIG. 4 shows the B-cell sub-population CD20 which may be seen in the CD19 gated density plot in the ‘all panel’ experiment (left) in the same distribution as the ungated B-cell only panel (right). Granulocyte markers CD11b and CD14 may be identified and similarly distinguished from each other in the ‘all panel’ experiment and in the ‘Granulocyte only’ panel as shown in FIG. 5 left and right plots respectively. In FIG. 6 it may be seen that NK cells may be viewed similarly in a single NK only panel or in the ‘all panel’ set. This invention provides a method to distinguish at least three different cell surface markers, CD25, CD20, CD14, labeled in this invention with antibodies conjugated to the same fluorescent dye (PE) by using appropriate gating of distinguishably labeled primary markers CD3, CD19, CD16 as seen in FIG. 7.

FIGS. 8-12 discussed below illustrate the categorization of primary populations (in this example MSCs and PBMCs) based on phenotypic properties such as cell size, autofluorescence, cell marker expression and combinations thereof. Approaches to categorizing primary populations based on such phenotypic properties find use in distinguishing detectable labels having substantially identical signals as illustrated by FIGS. 1-7 above, and in characterizing cell populations of co-cultures as illustrated by FIGS. 8-12 below.

Specifically, FIGS. 8-12 illustrate the discrimination of PBMCs and MSCs by flow cytometry based on size and cell signature. PBMCs and MSCs display significant differences in size and auto-fluorescence. Cells are therefore analyzed in a log scale and voltage settings are optimized based on MSCs showing higher autofluorescence (FIGS. 8 and 9). PBMCs can be discriminated based on size and exclusive expression of CD45 while MSCs can be discriminated based on size and expression ofCD73 and lack ofCD45 (FIG. 10). Using multicolor flow cytometry, aspects of the invention allows the determination that, for example, 1); IL-6 and CD274 expression by MSCs is upregulated in the presence of stimulated PBMCs; 2) IL-10-expressing CD14+CD206+ macrophages are upregulated in the presence of MSCs; 3) Stimulated PBMC proliferation and IFN-y expression are inhibited in the presence of MSCs (FIG. 11). Comparing functionally different MSCs using this methodology allowed the signature of immunosuppressive MSCs to be defined. Data generated using aspects of this invention suggest that adipose-derived MSCs expressing high levels of CD54 and IL-6 are better at inhibiting T-cell activation than bone marrow-derived MSCs expressing low levels of CD54 and IL-6 (FIG. 12). This signature could be used to screen MSCs prior to use in clinical settings to predict their immunosuppressive efficacy.

FIG. 8 shows differences in cell size and internal composition between PBMCs and MSCs as measured by forward scatter (FSC) and side scatter (SSC). Due to the difference in size, PBMCs and MSCs could not be simultaneously detected in linear scale. Using the setting optimized for PBMCs detection, MSCs would be out of scale (blue arrow). Using the setting optimized for MSC detection, it would not be possible to resolve PBMCs (green arrow). Use of log scale allows for the simultaneous detection and resolution of PBMCs and MSCs.

FIG. 9 shows differences in auto-fluorescence levels between PBMCs (P1) and MSCs (P2) as measured in a number of channels. MSCs are shown to be more auto-fluorescent than PBMCs. Voltage settings based on PBMCs auto-fluorescence were found to bring positive signal coming from MSCs out of scale. Therefore, voltages were set based on the cells with higher auto-fluorescence, in this case MSCs. Lower wavelength channels such as Alexa Fluor 488-A allowed for better separation as compared to higher wavelength channels such as Alexa Fluor 700-A. FIG. 10 shows discrimination of PBMCs and MSCs based on cell size as measured by light scatter and cell signature (e.g., surface marker expression). PBMCs and MSCs alone are initially analyzed to define the gating strategy for each population based on cell size and signature. PBMCs are smaller than MSCs and homogeneously express CD45 but not CD73 (left column). MSCs are bigger than PBMCs and almost exclusively express CD73 but not CD45 (middle column). Therefore PBMCs and MSCs can be discriminated based on size and signature (right column). FIG. 11 shows characterization of lymphocytes, monocytes and MSCs discriminated based on size and signature. FSC by SSC density plots on a log scale allow for resolution of lymphocytes, monocytes, and MSCs. Clear separation of cell types is achieved by further discriminating cell types based on their unique phenotype: CD45+CD14+CD3-monocytes, CD45+CD3+CD14-lymphocytes and CD73+CD45-MSCs (top panel). Each population is then interrogated using different combinations of antibodies (bottom panel).

FIG. 12 shows phenotypic and functional differences between adipose-derived and bone marrow-derived MSCs. Adipose-derived MSCs express high levels of CD 54 and IL-6 in resting conditions, i.e., when cultivated alone. Bone marrow-derived MSCs express lower levels of CD 54 and IL-6 in resting conditions. When co-cultured with stimulated PBMCs, CD54 hi IL-6 hi adipose MSCs exhibit higher immunosuppressive activity than CD54 low IL-6 low bone marrow MSCs, as showed by reduced levels of IFN-γ and reduced proliferation as compared to stimulated PBMCs in the absence of any MSCs.

TABLE 2
‘All Panel’ Labeling Spreadsheet

| | Primary Marker | | | |
|---|---|---|---|---|
| | T cell | B cell | NK | Granulocyte |
| | Primary Labels | | | |
| | CD3-ALX- | CD19- | | |
| Secondary Labels | 700 | V500 | CD56-APC | CD16-FITC |
| V450 | CD8 | | | |
| APCYCY7 | CD4 | | | CD11b |
| PE | CD25 | CD20 | CD337 | CD14 |

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of identifying sub-populations of cells in a cellular sample, the method comprising:
categorizing cells of the cellular sample into at least a first and second population based on a first phenotypic property; and
sub-categorizing each of the first and second populations into sub-populations of cells based on a second and third phenotypic property using X detectable labels providing Y distinct signals, wherein X>Y, to identify sub-populations of cells in the cellular sample.

2. The method of claim 1, further comprising distinguishing detectable labels providing a substantially identical signal based on the categorization of the cells.

3. The method of claim 1, further comprising detecting the Y distinct signals by flow cytometry.

4. The method of claim 1, wherein a data processing unit implements the step of identifying the second and third phenotypic property.

5. The method of claim 1, wherein the first phenotypic property is cell size.

6. The method of claim 5, wherein cell size is identified using forward scatter (FSC).

7. The method of claim 5, wherein cell size is identified using axial light loss (ALL).

8. The method of claim 1, wherein the first phenotypic property is cell granularity.

9. The method of claim 8, wherein cell granularity is identified using side scatter (SSC).

10. The method of claim 1, wherein the first phenotypic property is cell autofluorescence.

11. The method of claim 1, wherein the first phenotypic property is expression of a cellular marker.

12. The method of claim 11, wherein the expression of the cellular marker is identified using a detectable label that specifically binds to the cellular marker.

13. The method of claim 1, wherein the categorization of cells in the cellular sample into at least the first and second populations is based on the first phenotypic property and an additional phenotypic property.

14. (canceled)

15. The method of claim 1, wherein each of the X detectable labels comprises a binding domain and a label domain.

16-25. (canceled)

26. A labeled cellular sample, the sample comprising:
cells,
a first detectable label that specifically binds to a first cellular marker; and
a second detectable label that specifically binds to a second cellular marker;
wherein the first and second detectable labels provide a substantially identical signal.

27-39. (canceled)

40. A flow cytometry system comprising:
a flow cytometer configured to produce a data set;
a data processing unit; and
a memory storing a module for execution by the data processing unit, wherein the module is configured to transform the data set from a number (X) of signal sets to a number (Y) of marker density sets, wherein Y>X.

41-60. (canceled)

61. A module for execution by a data processing unit of a flow cytometry system, the module configured to transform the data set from a number (X) of signal sets to a number (Y) of marker density sets, wherein Y>X.

62-67. (canceled)

68. A kit comprising:
a first detectable label that specifically binds to a first cellular marker; and
a second detectable label that specifically binds to a second cellular marker;
wherein the first and second detectable labels provide a substantially identical signal.

69-75. (canceled)
